(19) United States
(12) Patent Application Publication
(10) Pub. No.: US 2026/0167942 A1
(43) Pub. Date: Jun. 18, 2026

(54) ADJUVANT FOR INTRADERMAL IMMUNIZATION AND USE THEREOF

(71) Applicants: JIANGSU ACADEMY OF AGRICULTURAL SCIENCES, Nanjing (CN); NANJING NATIONAL BIOTECHNOLOGY RESEARCH INSTITUTE CO., LTD., Nanjing (CN); GUOTAI (TAIZHOU) CENTER OF TECHNOLOGY INNOVATION FOR VETERINARY BIOLOGICALS, TAIZHOU, Taizhou (CN)

(72) Inventors: Bihua DENG, Nanjing (CN); Yu LU, Nanjing (CN); Haiyan WANG, Nanjing (CN); Wenzhu YIN, Nanjing (CN); Fang MA, Nanjing (CN); Yalu ZHU, Nanjing (CN); Yujie ZHOU, Nanjing (CN); Hai XU, Nanjing (CN); Qiurong QI, Nanjing (CN)

(73) Assignees: JIANGSU ACADEMY OF AGRICULTURAL SCIENCES, Nanjing (CN); NANJING NATIONAL BIOTECHNOLOGY RESEARCH INSTITUTE CO., LTD., Nanjing (CN); GUOTAI (TAIZHOU) CENTER OF TECHNOLOGY INNOVATION FOR VETERINARY BIOLOGICALS, TAIZHOU, Taizhou (CN)

(21) Appl. No.: 19/536,292

(22) Filed: Feb. 11, 2026

Related U.S. Application Data
(63) Continuation of application No. PCT/CN2024/125186, filed on Oct. 16, 2024.

(30) Foreign Application Priority Data
Sep. 24, 2024 (CN) 202411333683.2

(51) Int. Cl. C12N 7/00; A61K 39/00; A61K 39/21; A61K 45/06; C07K 14/005

(52) U.S. Cl. CPC C12N 7/00; A61K 39/00; A61K 39/21; A61K 45/06; C07K 14/005; A61K 2039/521; A61K 2039/552

(57) ABSTRACT

An adjuvant for intradermal immunization and a use thereof are provided. The adjuvant includes the following components in parts by mass: 10 parts to 25 parts of squalene, 10 parts to 25 parts of sea buckthorn oil, 25 parts to 35 parts of caprylocaproyl polyoxyl-8 glycerides, and 25 parts to 45 parts of Tween 80. The adjuvant for intradermal immunization has a low viscosity, is favorable for injection and absorption, and can significantly enhance the immunization efficacy of a vaccine.

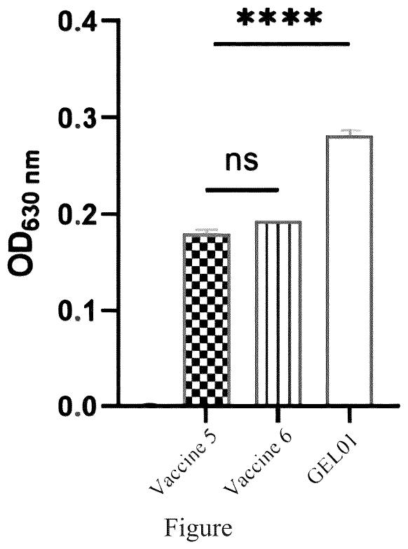

### CROSS REFERENCE TO THE RELATED APPLICATIONS

This application is a continuation application of International Application No. PCT/CN2024/125186, filed on Oct. 16, 2024, which is based upon and claims priority to Chinese Patent Application No. 202411333683.2, filed on Sep. 24, 2024, the entire contents of which are incorporated herein by reference.

### TECHNICAL FIELD

The present disclosure belongs to the field of pharmaceutical preparations, and specifically relates to an adjuvant for intradermal immunization and a use thereof.

### BACKGROUND

Intradermal immunization is a novel approach for animal vaccination. The skin is the outermost organ of the body, has a large surface area, and is well-suited for immunization and observation. Because the skin is relatively distant from vital organs of the body, intradermal immunization demonstrates a high level of safety. The skin and mucosa are frequently exposed to various foreign substances and possess a strong ability to recognize and process antigens. As a result, the skin and mucosa are ideal sites for vaccine administration. Additionally, the skin is rich in immune cells capable of generating an effective immune response to an administered vaccine. The use of intradermal immunization can significantly reduce the vaccine (antigen) dosage tenfold. Intradermal immunization is not only a theoretically ideal choice but has also been validated in practice. For example, the Bacille Calmette-Guérin (BCG) vaccine for preventing tuberculosis is administered intradermally. Intradermal immunization has been adopted in many countries worldwide. Intradermal immunization has also been utilized for rabies vaccination, which further demonstrates the efficacy and safety of intradermal vaccine immunization. In summary, due to its multifaceted advantages, intradermal immunization is regarded as an ideal vaccine administration route.

However, in current intradermal immunization practices of veterinary vaccines, the high viscosity of common adjuvants, such as an oil-based adjuvants, leads to issues such as increased difficulty of injection and impaired absorption of vaccine components in animals, which compromises both the immunization efficacy of a vaccine and the convenience of vaccination.

### SUMMARY

An objective of the present disclosure is to provide an adjuvant for intradermal immunization. This adjuvant has a low viscosity, is favorable for injection and absorption, and can significantly enhance the immunization efficacy of a vaccine.

Another objective of the present disclosure is to provide a use of the adjuvant.

The objectives of the present disclosure are achieved through the following technical solutions:

An adjuvant for intradermal immunization is provided, including the following components in parts by mass:
10 parts to 25 parts of squalene,
10 parts to 25 parts of sea buckthorn oil,
25 parts to 35 parts of caprylocaproyl polyoxyl-8 glycerides, and
25 parts to 45 parts of Tween 80.

In a preferred technical solution, the adjuvant includes the following components in parts by mass:
15 parts to 20 parts of the squalene,
15 parts to 20 parts of the sea buckthorn oil,
28 parts to 32 parts of the caprylocaproyl polyoxyl-8 glycerides, and
30 parts to 40 parts of the Tween 80.

The present disclosure also provides a use of the adjuvant in preparation of an inactivated porcine pseudorabies virus (PRV) vaccine.

In the present disclosure, the inactivated PRV vaccine is a vaccine for intradermal immunization.

In the present disclosure, the use includes: mixing the adjuvant and an antigen in a volume ratio of 1:(8-10), and conducting emulsification to produce the vaccine.

Compared with the existing adjuvants, the present disclosure offers the following beneficial effects: The adjuvant of the present disclosure is prepared by mixing immunostimulants (sea buckthorn oil and squalene) with surfactants (caprylocaproyl polyoxyl-8 glycerides and Tween 80). The adjuvant can be mixed with an inactivated antigen through manual shaking, magnetic stirring, or mechanical stirring to produce a stable oil-in-water (O/W) emulsion vaccine. A preparation process of the adjuvant does not require precision instruments, is easy to implement, and has a low production cost.

Additionally, all raw materials selected for the adjuvant are safe or low-toxicity substances that cause no toxic or side effects to animals.

A vaccine prepared using the adjuvant for intradermal immunization in the present disclosure has a low viscosity, and can be intradermally administered by a device. Moreover, the vaccine demonstrates advantages such as convenient injection, easy absorption, and prominent immunogenicity, and can significantly enhance the immunization efficacy.

### BRIEF DESCRIPTION OF THE DRAWINGS

FIGURE shows detection results of an antibody against PRV obtained using a blocking enzyme-linked immunosorbent assay (ELISA) antibody kit on day 28 d after immunization, where GEL01 represents control vaccine 1, ns indicates no significant difference, and **** indicates an extremely significant difference with p<0.001; and an OD value of an antibody detected during ELISA is inversely proportional to a titer of the antibody.

### DETAILED DESCRIPTION OF THE EMBODIMENTS

Caprylocaproyl polyoxyl-8 glycerides (Labrasol) purchased from Guangzhou Tianrun Pharmaceutical Co., Ltd., CAS No: 85536-07-8.

Propylene glycol dicaprate purchased from Guangzhou Tianrun Pharmaceutical Co., Ltd., CAS No: 56519-72-3.

Tween 80 (TWEEN™ 80-LQ-(RB)) purchased from Croda Chemicals (Shanghai) Co., Ltd., CAS No: 9005-65-6.

Sea buckthorn oil purchased from Shanghai Yuanye Biotechnology Co., Ltd. Cinnamon essential oil purchased from Taizhou Chundao Spice Co., Ltd.

Squalene purchased from Shanghai Yuanye Biotechnology Co., Ltd.

### Example 1 Preparation of O/W Emulsion Adjuvants

### Formula of Adjuvant 1:

40 g of sea buckthorn oil,
30 g of polyethylene glycol caprylic/capric glyceride caprylocaproyl polyoxyl-8 glycerides, and
30 g of Tween 80.

### Formula of Adjuvant 2:

40 g of squalene,
30 g of caprylocaproyl polyoxyl-8 glycerides, and
30 g of Tween 80.

### Formula of Adjuvant 3:

40 g of sea buckthorn oil,
10 g of squalene,
25 g of caprylocaproyl polyoxyl-8 glycerides, and
25 g of Tween 80.

### Formula of Adjuvant 4:

30 g of squalene,
20 g of sea buckthorn oil,
25 g of caprylocaproyl polyoxyl-8 glycerides, and
25 g of Tween 80.

### Formula of Adjuvant 5:

20 g of squalene,
20 g of sea buckthorn oil,
30 g of caprylocaproyl polyoxyl-8 glycerides, and
30 g of Tween 80.

### Formula of Adjuvant 6:

15 g of squalene,
15 g of sea buckthorn oil,
30 g of caprylocaproyl polyoxyl-8 glycerides, and
40 g of Tween 80.

### Formula of Adjuvant 7:

20 g of cinnamon essential oil,
10 g of vitamin E,
10 g of squalene,
30 g of caprylocaproyl polyoxyl-8 glycerides, and
30 g of propylene glycol dicaprate.

### Formula of Adjuvant 8:

40 g of cinnamon essential oil,
30 g of caprylocaproyl polyoxyl-8 glycerides, and
30 g of propylene glycol dicaprate.

### Formula of Adjuvant 9:

20 g of cinnamon essential oil,
10 g of squalene,
10 g of sea buckthorn oil,
30 g of caprylocaproyl polyoxyl-8 glycerides, and
30 g of propylene glycol dicaprate.

### Formula of Adjuvant 10:

20 g of squalene,
20 g of sea buckthorn oil,
30 g of caprylocaproyl polyoxyl-8 glycerides, and
30 g of glycerol formal.

Components were weighed according to the formulas of the adjuvants 1 to 10, mixed, and subjected to low-shear-force stirring or shaking for 5 min to 10 min to produce homogeneous systems. The homogeneous systems were each autoclaved at 115° C. for 20 min to produce the adjuvants 1 to 10, which were all O/W emulsions and stored at room temperature in the dark.

### Example 2 Inactivated PRV Vaccines, and Viscosity and Stability Testing Thereof

### 1. Inactivated PRV Vaccines

The inactivated PRV vaccines were prepared with the adjuvants in Example 1, respectively. Antigens in these vaccines were each a PRV strain PRV-NJ (disclosed in Chinese Invention Patent ZL201710943404.8).

Preparation of inactivated PRV vaccine 1: An inactivated PRV strain PRV-NJ solution (with a viral load of 10^{8.0} TCID50/mL before inactivation) and the adjuvant 1 were mixed in a volume ratio of 9:1 and stirred thoroughly to produce the inactivated PRV vaccine 1.

According to the preparation method of the inactivated PRV vaccine 1, the adjuvants 2 to 10 were each mixed with an inactivated PRV strain PRV-NJ solution (with a viral load of 10^{8.0} TCID50/mL before inactivation) in a volume ratio of 1:9, and stirring was conducted thoroughly to produce inactivated PRV vaccines 2 to 10. The numbers of the vaccines corresponded to the numbers of the adjuvants, respectively.

### 2. Viscosities of Vaccines

During a vaccine production process, it is crucial to control a viscosity of a vaccine in an appropriate range. The viscosity is a critical parameter measuring a quality of a vaccine, and directly affects the safety and efficacy of vaccine injection. A too-high viscosity may lead to severe issues such as skin necrosis at an injection site, compromising both the immunization effect of a vaccine and the health of a recipient. A viscosity of a vaccine is typically tested at a standard temperature of 25° C.

Viscosity measurement: A viscosity of each vaccine at 25° C. was measured using a DV2T cone-plate viscometer from Brookfield, USA. Test results were shown in Table 1. Viscosities of vaccines 1, 2, 5, and 6 were all lower than 2 mPa·s. Viscosities of vaccines 3 and 4 were 8.5 mPa·s and 4.4 mPa s, respectively. Viscosities of vaccines 7, 8, 9, and 10 were 10.5 mPa·s, 11.2 mPa·s, 10.2 mPa·s, and 10.6 mPa·s, respectively, which were significantly higher than those of other vaccines.

TABLE 1
Viscosity test results of vaccines

| Vaccine | Vaccine | Vaccine | Vaccine | Vaccine | Vaccine | Vaccine | Vaccine | Vaccine | Vaccine | Vaccine |
|---|---|---|---|---|---|---|---|---|---|---|
| No. | 1 | 2 | 3 | 4 | 5 | 6 | 7 | 8 | 9 | 10 |
| Viscosity | 1.5 | 1.6 | 8.5 | 4.4 | 1.4 | 1.3 | 10.5 | 11.2 | 10.2 | 10.6 |
| (mPa · s) | | | | | | | | | | |

### 3. Stability of Vaccines

Each vaccine was subjected to a centrifugation test and an accelerated aging stability test at 37° C.

Centrifugation test: For evaluating the stability of animal vaccines, observing the layering of an O/W emulsion vaccine under a centrifugal force is a simple and rapid method. The longer the time required for layering to occur, the better the stability of an oil emulsion. Conversely, if layering occurs rapidly and significantly, it indicates the poor stability of an oil emulsion. A specific process of a centrifugation test for a vaccine is as follows: 10 mL of the vaccine was taken and centrifuged at 3,000 r/min for 15 min. A volume of an aqueous phase separated at a bottom of a tube should not exceed 0.5 mL.

Accelerated aging stability test at 37° C.: The vaccines 1 to 10 were each placed in a 37° C. incubator and observed at a fixed time point daily. The separation of oil and aqueous phases was determined as emulsion breaking.

Results were shown in Table 2. The vaccines 1 to 10 were all O/W emulsion vaccines. After these vaccines were centrifuged, no aqueous phase was separated. Thus, the vaccines 1 to 10 all passed the centrifugation test. When the vaccines 1 to 10 were placed at 37° C. for 30 d, the separation of oil and aqueous phases was observed in vaccines 3 and 4 on day 5, indicating emulsion breaking. In contrast, there was no separation of oil and aqueous phases and no obvious appearance change in vaccines 1, 2, 5, 6, 7, 8, 9, and 10 during the 30 d storage period, indicating qualified thermal stability.

TABLE 2
Stability test results of vaccines

| | | Accelerated aging test |
|---|---|---|
| Group | Centrifugation results | results at 37° C. |
| Vaccine 1 | Qualified and no precipitation | No layering during the 30 d |
| | | storage period |
| Vaccine 2 | Qualified and no precipitation | No layering during the 30 d |
| | | storage period |
| Vaccine 3 | Qualified and no precipitation | Layering on day 5 |
| Vaccine 4 | Qualified and no precipitation | Layering on day 5 |
| Vaccine 5 | Qualified and no precipitation | No layering during the 30 d |
| | | storage period |
| Vaccine 6 | Qualified and no precipitation | No layering during the 30 d |
| | | storage period |
| Vaccine 7 | Qualified and no precipitation | No layering during the 30 d |
| | | storage period |
| Vaccine 8 | Qualified and no precipitation | No layering during the 30 d |
| | | storage period |
| Vaccine 9 | Qualified and no precipitation | No layering during the 30 d |
| | | storage period |
| Vaccine 10 | Qualified and no precipitation | No layering during the 30 d |
| | | storage period |

Vaccines 1, 2, 5, 6, 7, 8, 9, and 10 passed all tests, and vaccines 3 and 4 failed the accelerated aging stability test at 37° C., but passed the viscosity and centrifugation tests.

### Example 3 Immunization Safety Testing of Vaccines

The safety of intradermal immunization with the inactivated PRV vaccines 1, 2, 5, 6, 7, 8, 9, and 10 prepared in Example 2 was tested.

Piglet safety test: 45 PRV-negative piglets, approximately 30 days old, were selected and randomly divided into 9 groups. Necks of piglets in experimental groups were intradermally injected with the inactivated PRV vaccines 1, 2, 5, 6, 7, 8, 9, and 10 at a dose of 0.5 mL/piglet, respectively. Necks of piglets in a control group were intradermally injected with a same volume of phosphate buffered saline (PBS).

After intradermal immunization in a neck with each vaccine, a skin at an injection site was observed, and a post-injection adverse reaction was recorded. Criteria for determining the adverse reaction: (1) No adverse reaction: No change of a skin at an injection site (identical to normal skin). (2) Mild adverse reaction: Redness, swelling, or scabbing at an injection site. (3) Moderate adverse reaction: Nodule formation on a skin at an injection site. (4) Severe adverse reaction: Skin ulceration or worse at an injection site.

On day 1 to day 3 after vaccination, the absorption at an injection site was observed and recorded. Results were shown in Table 3. As shown in Table 3, after necks of PRV-negative piglets were intradermally injected with the vaccines 1, 2, 5, 6, 7, 8, 9, and 10, a wheal (a characteristic of intradermal injection) appeared. Vaccines 1, 2, 5, and 6 were absorbed by piglets within 1 d, and the wheal disappeared accordingly. Only piglet No. 2 vaccinated with the vaccine 1 required 2 d for absorption, and demonstrated a mild adverse reaction of slight redness and swelling. Among piglets vaccinated with vaccine 7, 2/5 underwent redness and swelling at an injection site that completely disappeared within 2 d to 3 d. Among piglets vaccinated with vaccine 8, 3/5 experienced redness and swelling that fully subsided within 2 d to 3 d, and 1/5 developed a persistent hard nodule. Among piglets vaccinated with vaccine 9, 2/5 suffered from redness and swelling that resolved completely within 2 d. Among piglets vaccinated with vaccine 10, 2/5 exhibited redness and swelling that resolved completely within 2 d. In the control group intradermally injected with PBS at a neck, PBS was absorbed within 2 h with no adverse reaction.

TABLE 3
Safety test results of intradermal immunization of piglets
with vaccines

| | | | | Ab- | Adverse reaction | | | |
|---|---|---|---|---|---|---|---|---|
| | Pig- | Age | | sorp- | | | Mo- | |
| | let | in | Injection | tion | | | de- | Se- |
| Group | No. | days | site | time | None | Mild | rate | vere |
| Vaccine | 1 | 30 | Intradermal | 1 d | + | − | − | − |
| 1 | | | injection | | | | | |
| | | | at a neck | | | | | |
| | 2 | 30 | Intradermal | 2 d | − | + | − | − |
| | | | injection | | | | | |
| | | | at a neck | | | | | |
| | 3 | 30 | Intradermal | 1 d | + | − | − | − |
| | | | injection | | | | | |
| | | | at a neck | | | | | |
| | 4 | 30 | Intradermal | 1 d | + | − | − | − |
| | | | injection | | | | | |
| | | | at a neck | | | | | |
| | 5 | 30 | Intradermal | 1 d | + | − | − | − |
| | | | injection | | | | | |
| | | | at a neck | | | | | |
| Vaccine | 6 | 30 | Intradermal | 1 d | + | − | − | − |
| 2 | | | injection | | | | | |
| | | | at aneck | | | | | |
| | 7 | 30 | Intradermal | 1 d | + | − | − | − |
| | | | injection | | | | | |
| | | | at a neck | | | | | |
| | 8 | 30 | Intradermal | 1 d | + | − | − | − |
| | | | injection | | | | | |
| | | | at a neck | | | | | |
| | 9 | 30 | Intradermal | 1 d | + | − | − | − |
| | | | injection | | | | | |
| | | | at a neck | | | | | |
| | 10 | 30 | Intradermal | 1 d | + | − | − | − |
| | | | injection | | | | | |
| | | | at a neck | | | | | |
| Vaccine | 11 | 30 | Intradermal | 1 d | + | − | − | − |
| 5 | | | injection | | | | | |
| | | | at a neck | | | | | |
| | 12 | 30 | Intradermal | 1 d | + | − | − | − |
| | | | injection | | | | | |
| | | | at a neck | | | | | |
| | 13 | 30 | Intradermal | 1 d | + | − | − | − |
| | | | injection | | | | | |
| | | | at a neck | | | | | |
| | 14 | 30 | Intradermal | 1 d | + | − | − | − |
| | | | injection | | | | | |
| | | | at a neck | | | | | |
| | 15 | 30 | Intradermal | 1 d | + | − | − | − |
| | | | injection | | | | | |
| | | | at a neck | | | | | |
| Vaccine | 16 | 30 | Intradermal | 1 d | + | − | − | − |
| 6 | | | injection at | | | | | |
| | | | a neck | | | | | |
| | 17 | 30 | Intradermal | 1 d | + | − | − | − |
| | | | injection | | | | | |
| | | | at a neck | | | | | |
| | 18 | 30 | Intradermal | 1 d | + | − | − | − |
| | | | injection | | | | | |
| | | | at a neck | | | | | |
| | 19 | 30 | Intradermal | 1 d | + | − | − | − |
| | | | injection | | | | | |
| | | | at a neck | | | | | |
| | 20 | 30 | Intradermal | 1 d | + | − | − | − |
| | | | injection | | | | | |
| | | | at a neck | | | | | |
| Vaccine | 21 | 30 | Intradermal | 1 d | + | − | − | − |
| 7 | | | injection | | | | | |
| | | | at a neck | | | | | |
| | 22 | 30 | Intradermal | 2 d | − | + | − | − |
| | | | injection | | | | | |
| | | | at a neck | | | | | |
| | 23 | 30 | Intradermal | 3 d | − | + | − | − |
| | | | injection | | | | | |
| | | | at a neck | | | | | |
| | 24 | 30 | Intradermal | 1 d | + | − | − | − |
| | | | injection | | | | | |
| | | | at a neck | | | | | |
| | 25 | 30 | Intradermal | 1 d | + | − | − | − |
| | | | injection | | | | | |
| | | | at a neck | | | | | |
| Vaccine | 26 | 30 | Intradermal | 2 d | − | + | − | − |
| 8 | | | injection | | | | | |
| | | | at a neck | | | | | |
| | 27 | 30 | Intradermal | 3 d | − | + | − | − |
| | | | injection | | | | | |
| | | | at a neck | | | | | |
| | 28 | 30 | Intradermal | 3 d | − | + | − | − |
| | | | injection | | | | | |
| | | | at a neck | | | | | |
| | 29 | 30 | Intradermal | Con- | − | − | + | − |
| | | | injection | tinuous | | | | |
| | | | at a neck | | | | | |
| | 30 | 30 | Intradermal | 1 d | + | − | − | − |
| | | | injection | | | | | |
| | | | at a neck | | | | | |
| Vaccine | 31 | 30 | Intradermal | 1 d | + | − | − | − |
| 9 | | | injection | | | | | |
| | | | at a neck | | | | | |
| | 32 | 30 | Intradermal | 2 d | − | + | − | − |
| | | | injection | | | | | |
| | | | at a neck | | | | | |
| | 33 | 30 | Intradermal | 1 d | + | − | − | − |
| | | | injection | | | | | |
| | | | at a neck | | | | | |
| | 34 | 30 | Intradermal | 1 d | + | − | − | − |
| | | | injection | | | | | |
| | | | at a neck | | | | | |
| | 35 | 30 | Intradermal | 2 d | − | + | − | − |
| | | | injection | | | | | |
| | | | at a neck | | | | | |
| Vaccine | 36 | 30 | Intradermal | 1 d | + | − | − | − |
| 10 | | | injection at | | | | | |
| | | | a neck | | | | | |
| | 37 | 30 | Intradermal | 2 d | − | + | − | − |
| | | | injection | | | | | |
| | | | at a neck | | | | | |
| | 38 | 30 | Intradermal | 2 d | − | + | − | − |
| | | | injection | | | | | |
| | | | at a neck | | | | | |
| | 39 | 30 | Intradermal | 1 d | + | − | − | − |
| | | | injection | | | | | |
| | | | at a neck | | | | | |
| | 40 | 30 | Intradermal | 1 d | + | − | − | − |
| | | | injection | | | | | |
| | | | at a neck | | | | | |
| Control | 41 | 30 | Intradermal | 2 h | + | − | − | − |
| | | | injection | | | | | |
| | | | at a neck | | | | | |
| | 42 | 30 | Intradermal | 2 h | + | − | − | − |
| | | | injection | | | | | |
| | | | at a neck | | | | | |
| | 43 | 30 | Intradermal | 2 h | + | − | − | − |
| | | | injection | | | | | |
| | | | at a neck | | | | | |
| | 44 | 30 | Intradermal | 2 h | + | − | − | − |
| | | | injection | | | | | |
| | | | at a neck | | | | | |
| | 45 | 30 | Intradermal | 2 h | + | − | − | − |
| | | | injection | | | | | |
| | | | at a neck | | | | | |

[]Notes:
In the table, “+” indicates presence and “−” indicates absence.

Additionally, after intradermal vaccination, all test piglets were monitored for 14 d in terms of feed and water intake, body weight loss, local reaction, allergic reaction, and body temperature. Results were shown in Table 4. As shown in Table 4, all test piglets exhibited normal feed and water intake and no body weight loss. Piglets vaccinated with vaccines 2, 7, 8, 9, and 10 showed redness, swelling, or a hard nodule at a vaccination site, but no abnormal reactions were observed in other groups. An average body temperature over 7 d was determined after vaccination, with a temperature difference of no more than 0.5° C.

TABLE 4
Safety test results of overdose vaccination of piglets

| | | | | | | Average body |
|---|---|---|---|---|---|---|
| | | | | | | temperature |
| | | Age | Feed and | Body | | over 7 d after |
| | | in | water | weight | Allergic | vaccination |
| Group | No. | days | intake | loss | reaction | (° C.) |
| Vaccine | 1 | 30 | Normal | None | None | 39.33 ± 0.29 |
| 1 | 2 | 30 | Normal | None | None | 39.80 ± 0.47 |
| | 3 | 30 | Normal | None | None | 39.29 ± 0.35 |
| | 4 | 30 | Normal | None | None | 39.41 ± 0.31 |
| | 5 | 30 | Normal | None | None | 39.25 ± 0.33 |
| Vaccine | 6 | 30 | Normal | None | None | 39.43 ± 0.29 |
| 2 | 7 | 30 | Normal | None | None | 39.36 ± 0.33 |
| | 8 | 30 | Normal | None | None | 39.30 ± 0.35 |
| | 9 | 30 | Normal | None | None | 39.27 ± 0.34 |
| | 10 | 30 | Normal | None | None | 39.22 ± 0.28 |
| Vaccine | 11 | 30 | Normal | None | None | 39.19 ± 0.30 |
| 5 | 12 | 30 | Normal | None | None | 39.27 ± 0.25 |
| | 13 | 30 | Normal | None | None | 39.53 ± 0.34 |
| | 14 | 30 | Normal | None | None | 39.25 ± 0.26 |
| | 15 | 30 | Normal | None | None | 39.24 ± 0.33 |
| Vaccine | 16 | 30 | Normal | None | None | 39.26 ± 0.25 |
| 6 | 17 | 30 | Normal | None | None | 39.28 ± 0.33 |
| | 18 | 30 | Normal | None | None | 39.35 ± 0.32 |
| | 19 | 30 | Normal | None | None | 39.30 ± 0.25 |
| | 20 | 30 | Normal | None | None | 39.26 ± 0.18 |
| Vaccine | 21 | 30 | Normal | None | None | 39.33 ± 0.36 |
| 7 | 22 | 30 | Normal | None | None | 39.25 ± 0.45 |
| | 23 | 30 | Normal | None | None | 39.33 ± 0.42 |
| | 24 | 30 | Normal | None | None | 39.32 ± 0.25 |
| | 25 | 30 | Normal | None | None | 39.30 ± 0.37 |
| Vaccine | 26 | 30 | Normal | None | None | 39.17 ± 0.43 |
| 8 | 27 | 30 | Normal | None | None | 39.28 ± 0.30 |
| | 28 | 30 | Normal | None | None | 39.33 ± 0.38 |
| | 29 | 30 | Normal | None | None | 39.33 ± 0.36 |
| | 30 | 30 | Normal | None | None | 39.25 ± 0.29 |
| Vaccine | 31 | 30 | Normal | None | None | 39.03 ± 0.29 |
| 9 | 32 | 30 | Normal | None | None | 39.32 ± 0.40 |
| | 33 | 30 | Normal | None | None | 39.35 ± 0.41 |
| | 34 | 30 | Normal | None | None | 39.01 ± 0.29 |
| | 35 | 30 | Normal | None | None | 39.32 ± 0.35 |
| Vaccine | 36 | 30 | Normal | None | None | 39.00 ± 0.33 |
| 10 | 37 | 30 | Normal | None | None | 39.25 ± 0.34 |
| | 38 | 30 | Normal | None | None | 39.31 ± 0.39 |
| | 39 | 30 | Normal | None | None | 39.37 ± 0.40 |
| | 40 | 30 | Normal | None | None | 39.19 ± 0.28 |
| Control | 41 | 30 | Normal | None | None | 39.31 ± 0.31 |
| | 42 | 30 | Normal | None | None | 39.29 ± 0.30 |
| | 43 | 30 | Normal | None | None | 39.35 ± 0.21 |
| | 44 | 30 | Normal | None | None | 39.22 ± 0.15 |
| | 45 | 30 | Normal | None | None | 39.35 ± 0.27 |

Note:
In this table, a body temperature (° C.) is expressed as mean ± standard deviation.

The inactivated PRV vaccines 1, 2, 5, 6, 7, 8, 9, and 10 prepared in Example 2 were tested for safety in pigs. Vaccines 1, 2, 5, and 6 met the current requirements for the safety of veterinary vaccines in the “Veterinary Pharmacopoeia of the People's Republic of China”, indicating that the intradermal immunization of adjuvants 1, 2, 5, and 6 were safe in pigs.

### Example 4 Intradermal Immunization Efficacy Testing of Inactivated PRV Vaccines

Preparation method of control vaccine 1: An inactivated PRV strain PRV-NJ solution (with a viral load of 10^{8.0} TCID50/mL before inactivation) and a GEL01 adjuvant (purchased from Seppic, France) were mixed in a volume ratio of 9:1 and stirred thoroughly to produce the control vaccine 1. According to test results, a viscosity of the control vaccine 1 was 4 mPa·s to 5 mPa·s, which was significantly higher than viscosities of the vaccines 5 and 6 in Example 2.

An immunization efficacy test was conducted by intradermally immunizing pigs with the inactivated PRV vaccines 5 and 6 prepared in Example 2 and the control vaccine 1. A specific process was as follows: 15 healthy and 30-day-old PRV-negative pigs were selected and randomly divided into 3 groups with 5 pigs in each group. Necks of pigs in groups 1 and 2 were intradermally injected with the vaccines 5 and 6 at a dose of 0.5 mL/pig, respectively. Necks of pigs in group 3 were intradermally injected with the control vaccine 1 at a dose of 0.5 mL/pig. After intradermal immunization in a neck with each vaccine, a skin at an injection site was observed, and a post-injection adverse reaction was recorded. Criteria for determining the adverse reaction: (1) No adverse reaction: No change of a skin at an injection site (identical to normal skin). (2) Mild adverse reaction: Redness, swelling, or scabbing at an injection site. (3) Moderate adverse reaction: Nodule formation on a skin at an injection site. (4) Severe adverse reaction: Skin ulceration or worse at an injection site. 28 d after immunization, blood was collected and serum was isolated. An anti-PRV antibody level was detected using an anti-PRV antibody ELISA kit (purchased from Wuhan Keqian Biology Co., Ltd.).

The vaccines 5 and 6 were absorbed within 1 d, a wheal disappeared, and there was no adverse reaction at an injection site. Among 5 pigs immunized with the control vaccine 1, 2 pigs developed a hard nodule at an injection site, indicating a moderate adverse reaction.

According to results of the immunization efficacy test in pigs, antibody titers in pigs injected with the vaccines 5 and 6 were significantly higher than antibody titers in pigs injected with the control vaccine 1 (the vaccine prepared with the imported adjuvant GEL01), and the vaccine 5 led to the optimal antibody titer. The main immunostimulant components in the vaccines 5 and 6 are sea buckthorn oil and squalene. The superior titers of the vaccines 5 and 6 to the control vaccine 1 after immunization prove that the combination of sea buckthorn oil and squalene exerts an immunostimulatory effect.

What is claimed is:

1. An adjuvant for intradermal immunization, comprising the following components in parts by mass:
10 parts to 25 parts of squalene,
10 parts to 25 parts of sea buckthorn oil,
25 parts to 35 parts of caprylocaproyl polyoxyl-8 glycerides, and
25 parts to 45 parts of Tween 80.

2. The adjuvant according to claim 1, comprising the following components in parts by mass:
15 parts to 20 parts of the squalene,
15 parts to 20 parts of the sea buckthorn oil,
28 parts to 32 parts of the caprylocaproyl polyoxyl-8 glycerides, and
30 parts to 40 parts of the Tween 80.

3. A method for preparing an inactivated porcine pseudorabies virus (PRV) vaccine, comprising using the adjuvant according to claim 1.

4. The method according to claim 3, wherein the inactivated PRV vaccine is a vaccine for the intradermal immunization.

5. The method according to claim 4, comprising: mixing the adjuvant and an antigen in a volume ratio of 1:(8-10), and conducting emulsification to produce the inactivated PRV vaccine.

6. The method according to claim 3, wherein the adjuvant comprises the following components in parts by mass:
15 parts to 20 parts of the squalene,
15 parts to 20 parts of the sea buckthorn oil,
28 parts to 32 parts of the caprylocaproyl polyoxyl-8 glycerides, and
30 parts to 40 parts of the Tween 80.

7. The method according to claim 6, wherein the inactivated PRV vaccine is a vaccine for the intradermal immunization.

8. The method according to claim 4, comprising: mixing the adjuvant and an antigen in a volume ratio of 1:(8-10), and conducting emulsification to produce the inactivated PRV vaccine;
wherein the adjuvant comprises the following components in parts by mass:
15 parts to 20 parts of the squalene,
15 parts to 20 parts of the sea buckthorn oil,
28 parts to 32 parts of the caprylocaproyl polyoxyl-8 glycerides, and
30 parts to 40 parts of the Tween 80.
